(19) United States
(12) Patent Application Publication
(10) Pub. No.: US 2018/0078673 A1
(43) Pub. Date: Mar. 22, 2018

(54) CELL CULTURE SUPPORT USING WATER-SOLUBLE POLYMER

(71) Applicant: AMOLIFESCIENCE CO., LTD., Seoul (KR)

(72) Inventors: In Yong SEO, Seoul (KR); Seung Hoon LEE, Paju-si (KR); Song Hee KOO, Seoul (KR); Ji Hyun LEE, Incheon (KR)

(21) Appl. No.: 15/559,237

(22) PCT Filed: May 10, 2016
(86) PCT No.: PCT/KR2016/004852

§ 371 Date: Sep. 18, 2017

(30) Foreign Application Priority Data
May 11, 2015 (KR) 10-2015-0065479

(51) Int. Cl. A61L 27/18; C12M 3/00; C12M 1/12; D01D 5/00

(52) U.S. Cl. CPC A61L 27/18; D01D 5/003; C12M 25/00; C12M 3/00

(57) ABSTRACT

Provided is a cell culture support using a water-soluble polymer, which is a support to which cells are attached to culture the cells, and which includes: a fibrous web having a plurality of pores in which fibers containing a water-soluble polymer and a synthetic polymer obtained by electrospinning are accumulated, and into which a culture solution is penetrated, and the water-soluble polymer of the fibers gradually dissolves in the culture solution so that the cells can be grown and eluted from the fibrous web, thereby gradually reducing the diameters of the fibers.

### TECHNICAL FIELD

The present invention relates to a cell culture support, and more particularly, to a cell culture support using a water-soluble polymer, which can elute cells to be grown, maximize a survival rate of cells by providing a culture environment familiar to cell culture, and grow cells in a desired shape and skeleton.

### BACKGROUND ART

Recently, as the use of cultured cells for the treatment of diseases has expanded, interest and research on cell culture have been increasing.

Cell culture is a technique to collect cells from living organisms and cultivate the cells in vitro. Cultured cells are used to treat diseases by differentiating them into various tissues of the body such as skin, organs, and nerves.

Such cell culture requires a culture support to provide a culture environment similar to the body.

Cells cultured on the culture support grow in an adhering state, and improving adhesion of the cells to the culture support can increase the survival rate of the cells.

Therefore, research and development of new culture supports to improve adhesion of cells and to further optimize the culture environment of cells are continuously being carried out.

Korean Patent Laid-open Publication No. 2007-0053443 discloses a method of producing a support made of a sponge-shaped fiber having a three-dimensional structure by performing a process of electrospinning a fiber spinning undiluted solution, but fibers of the support have a thread shape of a predetermined diameter and pores of the support are defined as spaces existing between the fibers.

Therefore, it is difficult for the cells to penetrate into the support via the fine pores of the support, and to grow. Thus, only the two-dimensional growth of the cells may be achieved. Therefore, there is a limit to the growth of the cells with a desired shape and skeleton, and there is a drawback that it is difficult to separate the cells from the support for differentiation after cell growth.

### DISCLOSURE

### Technical Problem

The present invention has been made in view of the above-described limit and drawback, and an object of the present invention is to provide a cell culture support using a water-soluble polymer capable of gradually dissolving the water-soluble polymer in a culture solution after cells are attached to a support, and eluting cells to be grown from a fibrous web.

Another object of the present invention is to provide a cell culture support using a water-soluble polymer capable of enabling the cells to penetrate into the fibrous web to then grow, thereby enabling the cells to grow without distorting the shapes and skeletons of the cells.

### Technical Solution

In order to achieve the above-mentioned object, there is provided a cell culture support using a water-soluble polymer according to an embodiment of the present invention, the cell culture support comprising: a fibrous web having a plurality of pores in which fibers containing a water-soluble polymer and a synthetic polymer obtained by electrospinning are accumulated, and into which a culture solution is penetrated, and the water-soluble polymer of the fibers gradually dissolves in the culture solution so that the cells can be grown and eluted from the fibrous web, thereby gradually reducing the diameters of the fibers.

In the cell culture support using the water-soluble polymer according to an embodiment of the present invention, the fibers may contain 10 wt % to 50 wt % of the water-soluble polymer.

In the cell culture support using the water-soluble polymer according to an embodiment of the present invention, the water-soluble polymer may be one or a mixture of two or more selected from among PVA (polyvinyl alcohol), PVP (polyvinyl pyrrolidone), polyethylene oxide (PEO), carboxyl methyl cellulose (CMC), starch, polyacrylic acid (PAA), and hyaluronic acid.

In the cell culture support using the water-soluble polymer according to an embodiment of the present invention, the diameter of the fiber may be 100 nm to 10 μm.

In the cell culture support using the water-soluble polymer according to an embodiment of the present invention, the synthetic polymer may be a biodegradable polymer.

In the cell culture support using the water-soluble polymer according to an embodiment of the present invention, the biodegradable polymer may be one of PLA, PLLA, PGA, PLGA, PCL and PDO.

In the cell culture support using the water-soluble polymer according to an embodiment of the present invention, the fibers may include an additive for hydrophilic treatment.

In the cell culture support using the water-soluble polymer according to an embodiment of the present invention, the hydrophilic treatment additive may be one of Tween 80, Pluronic, and PVP.

The cell culture support using the water-soluble polymer according to an embodiment of the present invention may further include a plurality of beads formed on the fibers in order to secure spaces in which the cells penetrate into the fibrous web and grow therein.

In the cell culture support using a water-soluble polymer according to an embodiment of the present invention, the fibrous web may be a web obtained by electrospinning a spinning solution in which the water-soluble polymer, the synthetic polymer and a solvent are mixed, and a viscosity of the spinning solution may be from 50 cps to 2000 cps.

In the cell culture support using the water-soluble polymer according to an embodiment of the present invention, the diameters of the beads may be larger than the diameters of the fibers.

In order to achieve the above-mentioned other object, there is provided a cell culture support using a water-soluble polymer according to an embodiment of the present invention, the cell culture support comprising: a first fibrous web made by accumulating first fibers containing a water-soluble polymer and a synthetic polymer, and formed with beads; a second fibrous web made by accumulating second fibers containing a water-soluble polymer and a synthetic polymer, and formed with beads, on the first fibrous web; and a third fibrous web made by accumulating third fibers containing a water-soluble polymer and a synthetic polymer, and formed with beads, on the second fibrous web.

In the cell culture support using the water-soluble polymer according to an embodiment of the present invention, the diameters of the second fibers may be smaller than the diameters of the first and third fibers.

In the cell culture support using the water-soluble polymer according to an embodiment of the present invention, the thicknesses of the first and third fibrous webs may be thinner than the thickness of the second fibrous web.

### Advantageous Effects

According to the present invention, there is an advantage that a fibrous web containing a water-soluble polymer is applied as a cell culture support, and the water-soluble polymer is slowly dissolved in a culture solution after the cells are attached to the cell culture support so that cells to be grown can be eluted from the fibrous web.

That is, since the cell culture support according to an embodiment of the present invention spontaneously elutes after the cells are grown, a process of separating the cells from the support for differentiation is unnecessary, and cell damage caused by cell separation in the support can be prevented.

According to the present invention, a cell survival rate can be maximized by implementing a cell culture support using a water-soluble polymer with a fibrous web having a structure most similar to an extracellular matrix (ECM) of the human body, thereby providing a familiar and suitable environment for cell culture.

According to the present invention, a plurality of beads suspended from the fibers of the fibrous web are formed to provide an enlarged space between the beads and the fibers and between the beads and the beads, so that the cultured cells can penetrate into the fibrous web to grow without distorting the shape and skeleton of the cells.

According to the present invention, a cell culture support is realized with a three-layered fibrous web structure to facilitate cell adhesion, to allow cells to penetrate into a laminated structure to grow, and to prevent the cells which penetrate into the laminated structure to grow from escaping from the bottom surface of the laminated structure, thereby providing the support on which cells can be grown with a desired shape and skeleton.

### BRIEF DESCRIPTION OF THE DRAWINGS

FIG. 1 is a schematic view showing a state where cells cultured in a cell culture support using a water-soluble polymer according to the present invention are eluted.

FIGS. 2A, 2B and 2C are views schematically showing a cell culture support in which the states of the fibers are changed by a water-soluble polymer dissolved over time according to the present invention.

FIG. 3 is a perspective view illustrating a state where beads are formed on a cell culture support using a water-soluble polymer according to the present invention.

FIGS. 4A and 4B illustrate SAM photographs of fibrous webs with and without beads according to the present invention.

FIG. 5 is a view schematically showing a state in which cells growing inside the cell culture support according to the present invention are infiltrated.

FIG. 6 is a schematic view for explaining an electrospinning apparatus for manufacturing a cell culture support using a water-soluble polymer according to the present invention.

FIG. 7 is a cross-sectional view of a cell culture support laminated according to the present invention.

FIG. 8 is a schematic cross-sectional view for explaining a method of manufacturing a cell culture support having a laminated structure according to the present invention.

### BEST MODE

Hereinafter, embodiments of the present invention will be described in detail with reference to the accompanying drawings.

In the present invention, a cell culture support is formed of a fibrous web having a plurality of pores and made by accumulating fibers obtained by electrospinning a spinning solution containing a mixture of a water-soluble polymer, a synthetic polymer and a solvent, and is characterized in that after cells are attached to the support, the water-soluble polymer is slowly dissolved in a culture solution to thereby realize the support capable of eluting the cells from the fibrous web.

In addition, according to an embodiment of the present invention, a fibrous web having a plurality of pores in which fibers containing a water-soluble polymer and a synthetic polymer obtained by electrospinning are accumulated and formed is provided as a support for cell culture, and has a structural feature in that a plurality of beads are formed on the fibers of the fibrous web so as to form spaces to allow the cells to be cultured to penetrate into the fibrous web and grow.

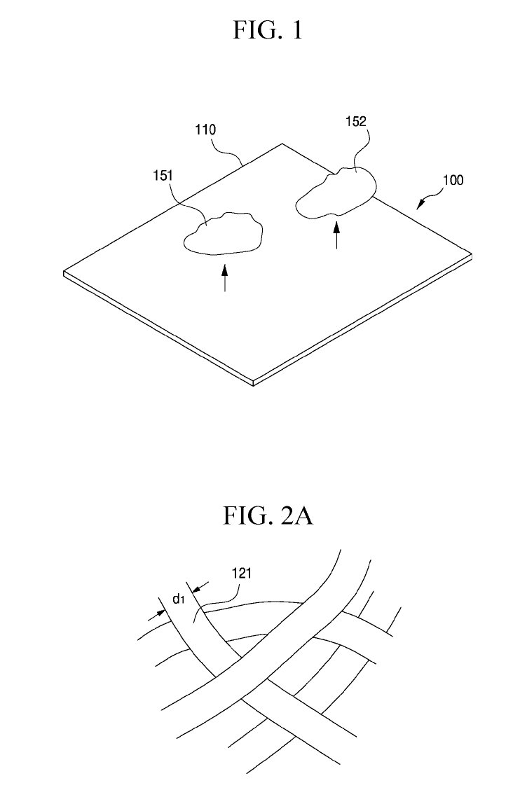

Referring to FIG. 1, a cell culture support using a water-soluble polymer according to an embodiment of the present invention is embodied as a fibrous web having a plurality of pores formed by accumulating fibers obtained by electrospinning a spinning solution containing a water-soluble polymer, a synthetic polymer, and a solvent.

The cell culture support according to the embodiment of the present invention cultivates cells in a state that the cells adhere to the support and are immersed in a culture solution, and the water-soluble polymer is slowly dissolved by the culture solution to reduce the diameters of the fibers, whereby the cells to be grown are eluted from the cell culture support.

That is, as the water-soluble polymer contained in the fibers of the cell culture support gradually dissolves, the adhesive force of the cells to the fibers is lowered. When most of the water-soluble polymer is dissolved and eluted from the fibers made of the water-soluble polymer and the synthetic polymer, and the cells can be eluted from the fibers.

In the total weight of the water-soluble polymer and the synthetic polymer, the water-soluble polymer is preferably contained in an amount of 10 wt % to 50 wt %. In other words, the fibers made of the water-soluble polymer and the synthetic polymer include 10 wt % to 50 wt % of the water-soluble polymer.

Here, when the content of the water-soluble polymer in the fibers is less than 10 wt %, the content of the fibers dissolved in the culture solution is so small that it is difficult to separate the cells from the fibers and the cells do not elute from the fibers. When the content of the water-soluble polymer exceeds 50 wt %, the content of the fibers is too large, and the content of the fibrous web is deformed, so that the cells cannot be cultured any more.

It is preferable that the diameters of the fibers of the fibrous web are 100 nm to 10 μm.

The water-soluble polymer may include one or a mixture of two or more selected from polyvinyl alcohol (PVA), polyvinyl pyrrolidone (PVP), polyethylene oxide (PEO), carboxyl methyl cellulose (CMC), starch, polyacrylic acid (PAA), and hyaluronic acid.

The synthetic polymer is capable of being electrospun and is not particularly limited as long as it can be dissolved in an organic solvent for electrospinning and is capable of forming fibers by electrospinning. For example, the synthetic polymer may include: polyvinylidene fluoride (PVdF), poly (vinylidene fluoride-co-hexafluoropropylene), perfluoropolymers, polyvinyl chloride or polyvinylidene chloride, and co-polymers thereof polyethylene glycol derivatives containing polyethylene glycol dialkylether and polyethylene glycol dialkyl ester; polyoxide containing poly (oxymethylene-oligo-oxyethylene), polyethylene oxide and polypropylene oxide; polyacrylonitrile co-polymers containing polyvinyl acetate, poly (vinyl pyrrolidone-vinyl acetate), polystyrene, polystyrene acrylonitrile co-polymers, polyacrylonitrile (PAN), and polyacrylonitrile methyl methacrylate co-polymers; and polymethyl methacrylate and polymethyl methacrylate co-polymers, and a mixture thereof.

Examples of the usable synthetic polymer may include: aromatic polyester such as polyamide, polyimide, polyamide-imide, poly (meta-phenylene iso-phthalamide), polysulfone, polyether ketone, polyethylene terephthalate, polytrimethylene terephthalate, and polyethylene naphthalate; polyphosphazenes such as polytetrafluoroethylene, polydiphenoxy phosphazene, and poly {bis [2-(2-methoxyethoxy) phosphazene]}; polyurethane co-polymers including polyurethane and polyether urethane; cellulose acetate, cellulose acetate butylrate, cellulose acetate propionate, and the like.

The solvent may employ at least one selected from the group consisting of DMAc (N, N-dimethyl acetoamide), DMF (N, N-dimethylformamide), NMP (N-methyl-2-pyrrolidinone), DMSO (dimethyl sulfoxide), THF (tetra-hydrofuran), EC (ethylene carbonate), DEC (diethyl carbonate), DMC (dimethyl carbonate), EMC (ethyl methyl carbonate), PC (propylene carbonate), water, acetic acid, formic acid, chloroform, dichloromethane, acetone, and isopropylalchol.

As described above, the cell culture support is made of a fibrous web having a plurality of pores formed by accumulating fibers obtained by electrospinning a spinning solution containing a water-soluble polymer, a synthetic polymer and a solvent, and the cell culture support cultures cells in a state where the cell culture support is immersed in a culture solution after the cells are attached to the cell culture support.

Here, the water-soluble polymer is slowly dissolved in the culture solution and the cells 151 and 152 to be grown are eluted from the fibrous web 110 constituting the cell culture support 100 as shown in FIG. 1.

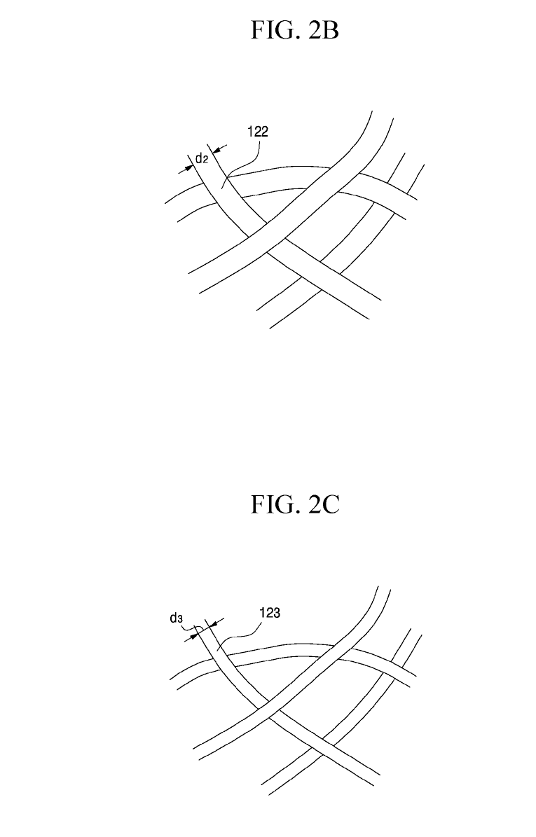

That is, as shown in FIG. 2A, cells adhere to the fibers 121 of the fibrous web 110 while the culture solution is immersed, and the cells are grown and the water-soluble polymer contained in the fibers 121 is dissolved in the culture solution over time. Accordingly, as shown in of FIG. 2B, the fiber 122 has the diameter d2 that becomes smaller than the diameter d1 of the fiber 121 in the state where the original cell is attached to the fiber 121.

Thereafter, the cells are further grown over further time, and the water-soluble polymer contained in the fiber 121 is mostly dissolved in the culture solution. As shown in FIG. 2C, the water-soluble polymer does not nearly remain in the fiber 123 and only the synthetic polymer is left. The diameter d3 of the fiber 123 is reduced to the maximum, so that the cells attached to the fiber 123 are eluted.

As described above, the cells adhering to and growing on the cell culture support 100 according to the embodiment of the present invention dissolve the water-soluble polymer in the culture solution in the fibers 121, 122 and 123, so that the cell adhesion to the cell culture support 100 is deteriorated, and the cells grow in a float state from the cell culture support 100, that is, in an eluted state.

Meanwhile, in some embodiment of the present invention, the synthetic polymer may be applied as a biodegradable polymer. The biodegradable polymer may be one of PLA (Poly Lactic Acid), PLLA (Poly (L-lactic acid)), PGA (Poly (glycolic acid)), PLGA (Poly (lactide-co-glycolide)), PCL (Polycaprolactone) and PDO (1,3-Propanediol).

That is, a fibrous web having a plurality of pores and made by accumulating fibers obtained by electrospinning a spinning solution containing a water-soluble polymer, a biodegradable polymer and a solvent, is used as a cell culture support, in which the water-soluble polymer is dissolved in the cell culture, and the cell culture support 100 composed of the biodegradable polymer is transplanted into the body after the cells are cultured to assist differentiation of the cells and is biodegraded and discharged from the inside of the body to the outside of the body at the end of the differentiation of the cells, to thereby realize the cell culture support that can be biodegraded after being transplanted into the body.

A biodegradable polymer is defined as a polymer that is completely decomposed into water and carbon dioxide, or water and methane gas by microorganisms such as bacteria, algae, and fungi in nature. It can be said that a biodegradable polymer is plastic whose physical and chemical structure is changed by organic matter such as bacteria in the natural world, so-called rotten plastic.

The biodegradable polymer has a characteristic that the rate of biodegradation greatly varies depending on the type of the polymer. Since the degradation rate can be controlled according to the composition ratio of the polymer degrading relatively quickly and the polymer degrading relatively slowly. Accordingly, there is also an advantage that the rate of degradation of the cell culture support can be controlled according to the cell differentiation rate after the cell culture support that is obtained by culturing the cells is transplanted into the body.

Therefore, the cell culture support using the biodegradable polymer-based water-soluble polymer according to some embodiment of the present invention a support that can make attachment of cells easily, can be transplanted into the body without side effects, and can be differentiated into organs or internal organs of necessary function after transplantation into the body, to then be biodegraded.

In addition, since the fibrous web used as a cell culture support in some embodiments of the present invention has the structure most similar to the extracellular matrix (ECM) of the human body, the support made of the fibrous web provides a familiar and suitable environment for cell culture to thereby maximize the survival rate of the cells.

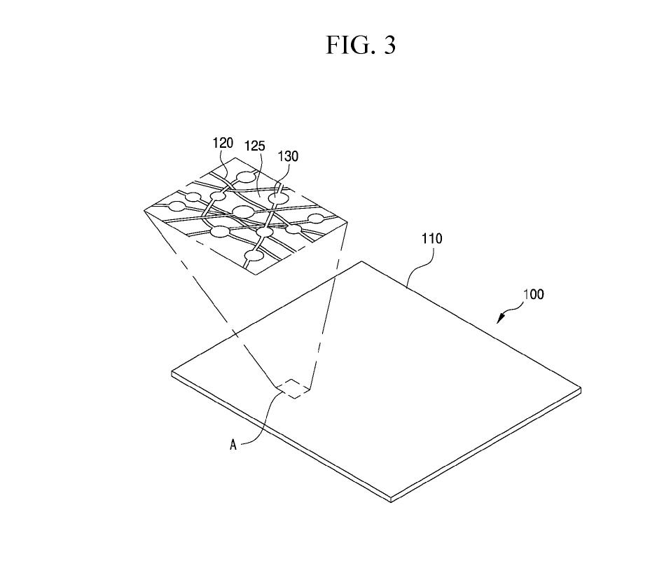

Referring to FIG. 3, a cell culture support 100 according to the embodiment of the present invention is a support to which cells are attached to culture the cells, and includes: a fibrous web 110 made by accumulating fibers 120 containing a water-soluble polymer and a synthetic polymer in which a plurality of pores 125 are formed; and a plurality of beads 130 formed on the fibers 120 to secure spaces for the cells to penetrate into the fibrous web 110 and grow therein.

When referring to an enlargement view of a region ‘A’ of a fibrous web 110 in FIG. 3, fibers 120 containing a water-soluble polymer and a synthetic polymer are unevenly accumulated to form a flat plate type fibrous web 110 and a plurality of pores 125 between the accumulated fibers 120.

Here, a plurality of beads 130 are formed on the fibers 120.

The diameters of the beads 130 are larger than the diameters of the fibers 120, and the beads 130 can be defined as an agglomerate of a biodegradable polymer. Here, at least one bead 130 is formed on each of all the fibers 120, or at least one bead 130 is formed on a part of all the fibers 120.

In some embodiments of the present invention, a water-soluble polymer, a synthetic polymer and a solvent are mixed to prepare a spinning solution, the spinning solution is electrospun from a nozzle of a spinning apparatus to be described later to form a fiber 120 in which the bead 130 is suspended, and the fiber 120 is accumulated, to produce a fibrous web 110 for a cell culture support 100.

Here, in some embodiments of the present invention, the viscosity of the spinning solution in which the water-soluble polymer, the synthetic polymer and the solvent are mixed is set to 50 cps to 2000 cps in order to realize the fibers having beads.

Here, if the viscosity of the spinning solution is less than 50 cps, the flow ability of the spinning solution is high and the droplet is sprayed from the nozzle of the spinning apparatus. If the viscosity of the spinning solution exceeds 2000 cps, the amount of an organic solvent in the spinning solution becomes small, and thus the flow ability of the spinning solution is low. In this case, only fibers are spun from the nozzle of the spinning apparatus.

The inventor(s) of the present invention confirmed through experiments that the formation of beads in the fibers 120 produced by electrospinning from the spinning nozzle is closely related to the viscosity of the spinning solution.

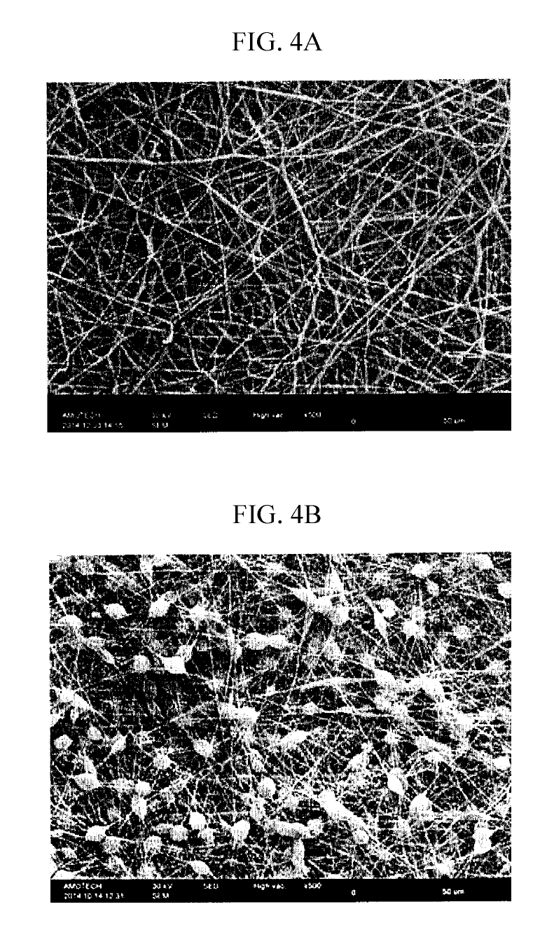

That is, the water-soluble polymer was excluded, the synthetic polymer was applied with a PLGA having a molecular weight of 130,000, and the PLGA and the solvent were mixed so as to have a viscosity of 2100 cps and electrospun. As a result, a fibrous web consisting of only fibers was produced as shown in FIG. 4A. However, the spinning solution in which the PLGA and the solvent were mixed was electrospun so as to have a viscosity of 260 cps in order to satisfy a viscosity range set in the embodiment of the present invention, to prepare a fibrous web in which fibers having beads were accumulated as shown in FIG. 4B.

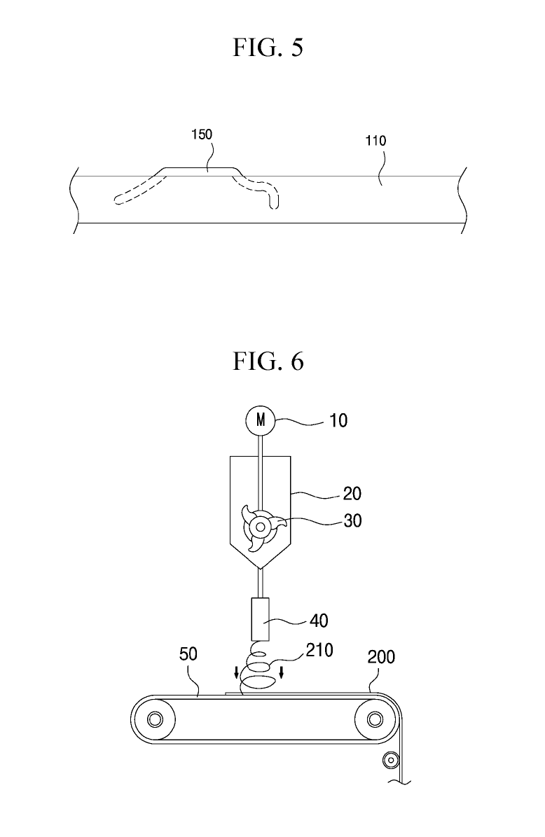

Accordingly, in some embodiments of the present invention, the cell culture support is embodied as a fibrous web containing a water-soluble polymer and a synthetic polymer, and a plurality of beads suspended from the fibers of the fibrous web are provided to form enlarged spaces (i.e. large pores) between a bead and a fiber, and between one bead and another bead. Accordingly, there is an advantage that the cells 150 cultured on the fibrous web 110 can penetrate into the fibrous web 110 and grow three-dimensionally as shown in FIG. 5.

That is, the fibrous web accumulated in the fibers only forms micropores between the fibers, but the fibrous web used as the cell culture support according to some embodiments of the present invention has pores between the bead and the fiber and between the beads. Therefore, the pores of the fibrous web according to some embodiments of the present invention in which beads are present become pores larger than the micropores formed between the fibers of the fibrous web in which beads are not present, thereby facilitating the penetration of the cells 150 to be grown.

Meanwhile, in some embodiments of the present invention, in order to impart hydrophilicity to fibers and beads constituting a fibrous web of a cell culture support, a water-soluble polymer, a synthetic polymer, an additive for hydrophilic treatment and a solvent are mixed to prepare a spinning solution, and the spinning solution is electrospun to accumulate fibers having beads having hydrophilicity to thereby produce a fibrous web.

Here, the additive for hydrophilic treatment may be one of Tween 80, Pluronic, and PVP.

The cell culture support is immersed in the culture solution, and the cells attached to the cell culture support grow by absorbing the nutrients from the culture solution. The cells can well adhere to a support having excellent hydrophilicity. Accordingly, according to some embodiments of the present invention, an additive for hydrophilic treatment is incorporated into the fibers and beads to thus realize a fibrous web having high hydrophilicity, to thereby provide an advantage of facilitating attachment of cells.

FIG. 6 is a schematic view illustrating an electrospinning apparatus for preparing a cell culture support according to an embodiment of the present invention.

Referring to FIG. 6, an electrospinning apparatus for producing a cell culture support according to an embodiment of the present invention is characterized in that a stirring tank 20 for supplying a stirred spinning solution is connected to a spinning nozzle 40, a grounded collector 50 in the form of a conveyor that moves at a constant speed is placed in a lower portion of the electrospinning apparatus and spaced from the spinning nozzle 40, and the spinning nozzle 40 is connected to a high voltage generator.

Here, a water-soluble polymer, a synthetic polymer, and a solvent are mixed with a stirrer 30 to prepare a spinning solution. Here, a pre-mixed spinning solution may be used before being put into the electrospinning apparatus without mixing a biodegradable polymer and a solvent in the stirrer 30.

Thereafter, when a high voltage electrostatic force is applied between the collector 50 and the spinning nozzle 40, the spinning solution is spun by the spinning nozzle 40 into the ultrafine fibers 210 to then be emitted to the collector 50. The fibers 210 are accumulated to the collector 50 to form the fibrous web 200 of the cell culture support.

More specifically, the spinning solution discharged from the spinning nozzle 40 is discharged as the ultrafine fibers 210 while passing through the spinning nozzle 40 charged by the high voltage generator, and the ultrafine nanofibers 210 are sequentially laminated on the grounded collector 50 provided in the form of a conveyor moving at a certain speed to form the fibrous web 200 of the cell culture support.

Meanwhile, the cell culture support 100 according to some embodiments of the present invention can be realized as a laminated structure in which a plurality of fibrous webs 110 are laminated in which each fibrous web is formed by accumulating fibers a water-soluble polymer and a synthetic polymer and forming a plurality of beads on the fibers.

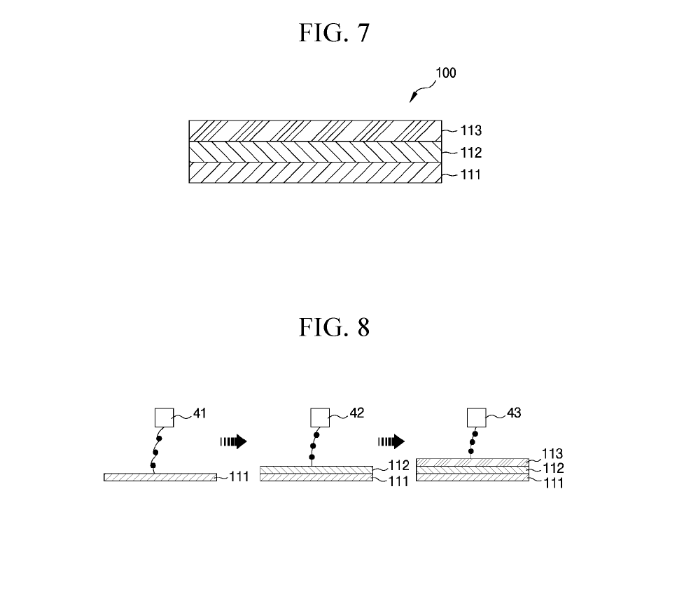

As an example, as shown in FIG. 7, the cell culture support 100 having a laminated structure may include a first fibrous web 111 made by accumulating first fibers having beads; a second fibrous web 112 made by accumulating second fibers having beads on the first fibrous web 111; and a third fibrous web 113 made by accumulating third fibers having beads on the second fibrous web 112.

In the cell culture support 100 having such a three-layer laminated structure, it is preferable that the second fibers have smaller diameters than the first and third fibers.

That is, the third fibrous web 113 is laminated on the second fibrous web 112, and cells are attached to the third fibrous web 113 and cultured. Here, the diameters of the third fibers of the third fibrous web 113 are made smaller than the diameters of the second fibers of the second fibrous web 112, thereby widening the surface area to which the cells are attached so that the cells can adhere well.

Also, in some embodiments of the present invention, it is preferable that the thickness of each of the first and third fibrous webs 111 and 113 is thinner than the thickness of the second fibrous web 112.

The diameters of the second fibers of the second fibrous web 112 are larger than the diameters of the third fibers of the third fibrous web 113 and the space of the second fibrous web 112 is larger than the space of the third fibrous web 113, to thereby promote penetration of grown cells into the second fibrous web 112. The diameters of the first fibers of the first fibrous web 111 are less than the diameters of the second fibers of the second fibrous web 112 and the space of the first fibrous web 111 is narrower than the space of the second fibrous web 112 so that cells that penetrate and grow into the second fibrous web 112 are infiltrated into the first fibrous web 111 and can be prevented from growing on the bottom surface of the first fibrous web 111.

Therefore, the cell culture support 100 according to some embodiments of the present invention is realized in a structure in which the fibrous web is laminated in three layers, and thus there are several advantages of facilitating cell adhesion, allowing the cells to penetrate into the laminate structure and grow therein, preventing the cells infiltrated and grown into the laminate structure from escaping from the bottom surface of the laminated structure, so that the grown cells can have desired shapes and skeletons without being distorted.

FIG. 8 is a schematic cross-sectional view for explaining a method of manufacturing a cell culture support having a laminated structure according to the present invention.

The cell culture support having a laminated structure is formed by accumulating fibers having beads discharged from first to third spinning nozzles 41, 42, and 43.

The spinning solution in which the water-soluble polymer, the synthetic polymer and the solvent are mixed is supplied to the first to third spinning nozzles 41, 42 and 43 to discharge fibers having beads, and the first to third spinning nozzles 41, 42 and 43 are sequentially placed on the collector 50 moving at a constant speed of the above-described electrospinning apparatus.

First, after the first fibrous web 111 is formed by discharging the first fibers having beads from the first spinning nozzle 41, the first fibrous web 111 is moved to the lower portion of the second spinning nozzle 42. Then, the second fibrous web 112 is laminated on the first fibrous web 111 by discharging the second fibers having beads on the first fibrous web 111 by the second spinning nozzle 42.

Then, the second fibrous web 112 moves to the lower portion of the third spinning nozzle 43. Then, the third spinning nozzle 43 discharges the third fibers having beads on the upper portion of the second fibrous web 112, so that the third fibrous web 113 is laminated on the second fibrous web 112.

While the present invention has been particularly shown and described with reference to exemplary embodiments thereof, by way of illustration and example only, it is clearly understood that the present invention is not to be construed as limiting the present invention, and various changes and modifications may be made by those skilled in the art within the protective scope of the invention without departing off the spirit of the present invention.

### INDUSTRIAL APPLICABILITY

The present invention is applied to a cell culture support using a water-soluble polymer, which can elute cells to be grown, maximize a survival rate of cells by providing a culture environment familiar to cell culture, and grow cells in a desired shape and skeleton.

What is claimed is:

1. A cell culture support using a water-soluble polymer, the cell culture support, which is a support to which cells are attached to culture the cells, the cell culture support comprising:
a fibrous web having a plurality of pores in which fibers containing a water-soluble polymer and a synthetic polymer obtained by electrospinning are accumulated, and into which a culture solution is penetrated, and
the water-soluble polymer of the fibers gradually dissolves in the culture solution so that the cells can be grown and eluted from the fibrous web, thereby gradually reducing the diameters of the fibers.

2. The cell culture support using a water-soluble polymer of claim 1, wherein the fibers contain 10 wt % to 50 wt % of the water-soluble polymer.

3. The cell culture support using a water-soluble polymer of claim 1, wherein the water-soluble polymer is one or a mixture of two or more selected from among PVA (polyvinyl alcohol), PVP (polyvinyl pyrrolidone), polyethylene oxide (PEO), carboxyl methyl cellulose (CMC), starch, polyacrylic acid (PAA), and hyaluronic acid.

4. The cell culture support using a water-soluble polymer of claim 1, wherein the diameter of each of the fibers is 100 nm to 10 μm.

5. The cell culture support using a water-soluble polymer of claim 1, wherein the synthetic polymer is a biodegradable polymer.

6. The cell culture support using a water-soluble polymer of claim 5, wherein the biodegradable polymer is one of PLA, PLLA, PGA, PLGA, PCL and PDO.

7. The cell culture support using a water-soluble polymer of claim 1, wherein the fibers an additive for hydrophilic treatment.

8. The cell culture support using a water-soluble polymer of claim 7, wherein the hydrophilic treatment additive is one of Tween 80, Pluronic, and PVP.

9. The cell culture support using a water-soluble polymer of claim 1, further comprising a plurality of beads formed on the fibers in order to secure spaces in which the cells penetrate into the fibrous web and grow therein.

10. The cell culture support using a water-soluble polymer of claim 9, wherein the fibrous web is a web obtained by electrospinning a spinning solution in which the water-soluble polymer, the synthetic polymer and a solvent are mixed, and a viscosity of the spinning solution is from 50 cps to 2000 cps.

11. The cell culture support using a water-soluble polymer of claim 9, wherein the diameters of the beads are larger than the diameters of the fibers.

12. A cell culture support using a water-soluble polymer, the cell culture support comprising:
a first fibrous web made by accumulating first fibers containing a water-soluble polymer and a synthetic polymer, and formed with beads;
a second fibrous web made by accumulating second fibers containing a water-soluble polymer and a synthetic polymer, and formed with beads, on the first fibrous web; and
a third fibrous web made by accumulating third fibers containing a water-soluble polymer and a synthetic polymer, and formed with beads, on the second fibrous web.

13. The cell culture support using a water-soluble polymer of claim 12, wherein the diameters of the second fibers are smaller than the diameters of the first and third fibers.

14. The cell culture support using a water-soluble polymer of claim 12, wherein the thicknesses of the first and third fibrous webs are thinner than the thickness of the second fibrous web.
